(19) United States
(12) Patent Application Publication
(10) Pub. No.: US 2026/0325070 A1
(43) Pub. Date: Oct. 22, 2026

(54) WEARABLE ELECTRONIC DEVICE INCLUDING SENSOR ASSEMBLY

(71) Applicant: Samsung Electronics Co., Ltd., Suwon-si (KR)

(72) Inventors: Jungsoo KIM, Suwon-si (KR); Jaejong RYU, Suwon-si (KR)

(21) Appl. No.: 19/695,284

(22) Filed: Jun. 2, 2026

Related U.S. Application Data
(63) Continuation of application No. PCT/KR2024/096953, filed on Dec. 13, 2024.

(30) Foreign Application Priority Data
Dec. 26, 2023 (KR) 10-2023-0191835
Jan. 30, 2024 (KR) 10-2024-0014022

(51) Int. Cl. A61B 5/00; A61B 5/0205; A61B 5/117; G04G 17/04; G04G 17/08; G04G 21/02

(52) U.S. Cl. CPC A61B 5/681; A61B 5/0205; A61B 5/117; A61B 5/742; G04G 17/045; G04G 17/08; G04G 21/025

(57) ABSTRACT

An electronic device is provided. The electronic device includes a housing including a front plate being partially inclined, a rear plate facing in a direction opposite to the front plate, and a side member surrounding an inner space between the front plate and the rear plate, a molding member disposed between the front plate and the rear plate, the molding member dividing the inner space into a first inner space and a second inner space, a display disposed in the first inner space, and a sensor assembly non-overlapping with the display, the sensor assembly being disposed in the second inner space, wherein the front plate covers the display and the sensor assembly.

### CROSS-REFERENCE TO RELATED APPLICATION(S)

This application is a continuation application, claiming priority under 35 U.S.C. § 365(c), of an International application No. PCT/KR2024/096953, filed on Dec. 13, 2024, which is based on and claims the benefit of a Korean patent application number 10-2023-0191835, filed on Dec. 26, 2023, in the Ministry of Intellectual Property (MOIP), and of a Korean patent application number 10-2024-0014022, filed on Jan. 30, 2024, in the Ministry of Intellectual Property (MOIP), the disclosure of each of which is incorporated by reference herein in its entirety.

### BACKGROUND

### 1. Field

The disclosure relates to a wearable electronic device including a sensor assembly.

### 2. Description of Related Art

A portable electronic device, such as a smartphone, may provide various functions, including call functions, based on various types of applications. Recently, as wearable electronic devices have been widely distributed, a demand of consumers for wearable electronic devices that provide more various functions is increasing. A user may wear a wearable electronic device on at least a portion of the body, so that various functions provided by the wearable electronic device may be conveniently and easily provided.

For example, technologies for sensing biometric information of a user by using a wearable electronic device have been researched. A wearable electronic device may acquire biometric information of a user and may provide various functions for health care based on the acquired biometric information.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a wearable electronic device including a sensor assembly.

Additional aspects will be set forth in part in the description which follows and, in part, will be apparent from the description, or may be learned by practice of the presented embodiments.

In accordance with an aspect of the disclosure, an electronic device is provided. The electronic device includes a housing including a front plate being partially inclined, a rear plate facing in a direction opposite to the front plate, and a side member surrounding an inner space between the front plate and the rear plate, a molding member disposed between the front plate and the rear plate, the molding member dividing the inner space into a first inner space and a second inner space, a display disposed in the first inner space, and a sensor assembly non-overlapping with the display, the sensor assembly being disposed in the second inner space, wherein the front plate covers the display and the sensor assembly.

In accordance with another aspect of the disclosure, a wearable electronic device is provided. The wearable electronic device includes a housing including a front plate, a portion of which is inclined, a display covered by the front plate, the display being disposed within the housing, a sensor assembly covered by the front plate, the sensor assembly being disposed adjacent to the display, and a molding member disposed between the display and the sensor assembly, wherein the sensor assembly includes a first biometric sensor unit disposed within the housing, the first biometric sensor unit non-overlapping with the display, and a second biometric sensor unit non-overlapping with the display, the second biometric sensor unit including a first conductive member partially disposed on a first surface of the front plate.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment, according to an embodiment of the disclosure;

FIG. 2A is a perspective view of a front surface of an electronic device according to an embodiment of the disclosure;

FIG. 2B is a perspective view of a rear surface of the electronic device of FIG. 2A according to an embodiment of the disclosure;

FIG. 3 is an exploded perspective view illustrating an electronic device according to an embodiment of the disclosure;

FIG. 4 is a view illustrating a portion of a wearable electronic device according to an embodiment of the disclosure;

FIG. 5 is a view illustrating a housing and a fastening member of a wearable electronic device according to an embodiment of the disclosure;

FIG. 6 is a view illustrating a portion of a wearable electronic device according to an embodiment of the disclosure;

FIG. 7 is a view illustrating a cross section of a portion of the wearable electronic device illustrated in FIG. 6 according to an embodiment of the disclosure;

FIG. 8 is a view illustrating a cross section of a portion of a wearable electronic device according to an embodiment of the disclosure;

FIGS. 9A and 9B are views illustrating a method for manufacturing a wearable electronic device according to various embodiments of the disclosure;

FIG. 10A is a view illustrating a molding member included in a wearable electronic device according to an embodiment of the disclosure;

FIG. 10B is a view illustrating an apparatus for forming the molding member illustrated in FIG. 10A according to an embodiment of the disclosure;

FIG. 11 is a view illustrating a portion of a wearable electronic device according to an embodiment of the disclosure;

FIG. 12A is a view illustrating a state, in which a wearable electronic device is mounted on a body of a user according to an embodiment of the disclosure;

FIG. 12B is a conceptual view illustrating a method for acquiring biometric information through the wearable electronic device according to an embodiment of the disclosure; and

FIG. 13 is a conceptual view illustrating a method for acquiring biometric information through a wearable electronic device according to an embodiment of the disclosure.

Throughout the drawings, like reference numerals will be understood to refer to like parts, components, and structures.

### DETAILED DESCRIPTION

The following description with reference to accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

An embodiment of the disclosure described below provides a wearable electronic device that is capable of acquiring biometric information of a user by optical and electrical measurement methods, by including a sensor assembly that includes a first biometric sensor unit that does not overlap the display and is disposed in a housing, and a second biometric sensor unit that does not overlap the display and includes a conductive member that is partially formed on one surface of a front plate.

Furthermore, an embodiment of the disclosure provides a wearable electronic device that is capable of acquiring biometric information of a user by optical and electrical measurement methods, by including a front plate that includes a first window area and a second window area that extends obliquely from the first window area, a display that overlaps the first window area, and a sensor assembly that overlaps the second window area.

The objects according to various embodiments of the disclosure will be described as necessary in a process of describing the embodiments.

A wearable electronic device according to an embodiment is capable of acquiring accurate and precise biometric information because the wearable electronic device acquires biometric information of a user by optical and electrical measurement methods.

In a wearable electronic device according to an embodiment, because biometric information of a user may be acquired by using a first biometric sensor unit at a finger portion, at which a blood vessel is located toward a skin, a high signal quality of the biometric information (e.g., a signal-to-noise ratio (SNR)) may be secured.

Furthermore, a wearable electronic device according to an embodiment is capable of acquiring biometric information of a user with emission of a small amount of light, so that an advantage of a current consumption may be secured, and various wavelength bands of light may be utilized.

Furthermore, a wearable electronic device according to an embodiment is capable of providing comfort in a measurement posture because a first biometric sensor unit and a second biometric sensor unit may secure a degree of freedom from a key button.

Furthermore, a wearable electronic device according to an embodiment is capable of implementing a sense of design unity because a display and a sensor assembly are covered through a single front plate, and a fastening member is disposed adjacent to the front plate.

In addition, various purposes and effects provided by the wearable electronic device according to various embodiments may be mentioned according to embodiments of the detailed description.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi) chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

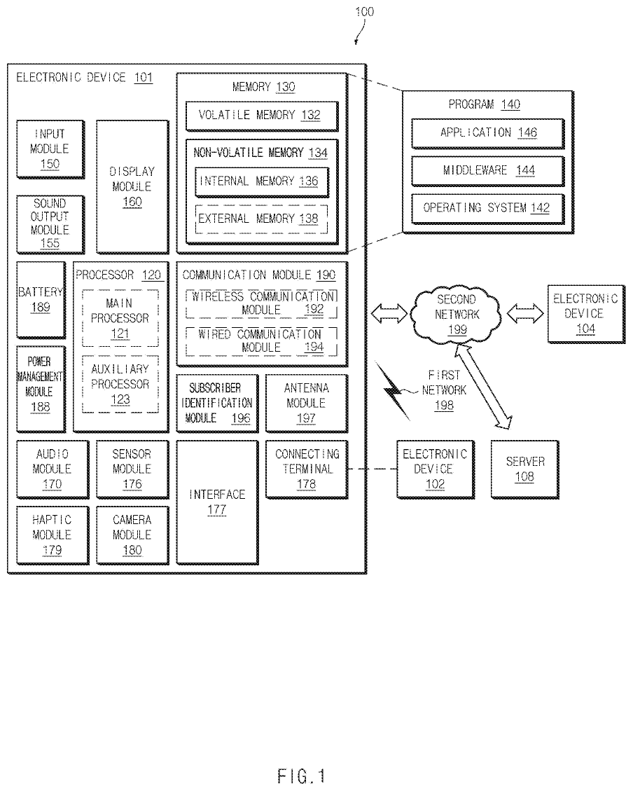

FIG. 1 is a block diagram illustrating an electronic device in a network environment according to an embodiment of the disclosure.

Referring to FIG. 1, an electronic device 101 (e.g., the electronic device 100 of FIG. 1) in a network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, a memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one (e.g., the connecting terminal 178) of the components may be omitted from the electronic device 101, or one or more other components may be added in the electronic device 101. In some embodiments, some of the components may be implemented as single integrated circuitry. For example, some (e.g., the sensor module 176, the camera module 180, or the antenna module 197) of the components may be integrated into a single component (e.g., the display module 160)).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may load a command or data received from another component (e.g., the sensor module 176 or the communication module 190) in a volatile memory 132, process the command or the data stored in the volatile memory 132, and store resulting data in a non-volatile memory 134. According to an embodiment, the processor 120 may include a main processor 121 (e.g., a central processing unit (CPU) or an application processor (AP)), and an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. When the electronic device 101 includes the main processor 121 and the auxiliary processor, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., a neural network processing device) may include a hardware structure specified for processing an artificial intelligence (AI) model. The AI model may be generated through machine learning. The learning may be performed by the electronic device 101 performing the AI, and may be performed through an additional server (e.g., the server 108). A learning algorithm may include, for example, a supervised learning algorithm, an unsupervised learning algorithm, a semi-supervised learning algorithm, or a reinforcement learning algorithm, but the disclosure is not limited thereto. The AI model may include a plurality of artificial neural network (ANN) layers. The ANN may include a deep neural network (DNN), a convolutional neural network (CNN), a recurrent neural network (RNN), a restricted boltzman machine (RBM), a deep belief network (DBN), a bidirectional recurrent deep neural network (BRDNN), a deep Q-networks or the combination of the above networks, but the disclosure is not limited thereto. The AI model may additionally or alternatively include a software structure, in addition to a hardware structure.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record, and the receiver may be used for incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or an external electronic device (e.g., the electronic device 102) (e.g., speaker of headphone) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). The communication module according to an embodiment may communicate with the external electronic device 104 via the first network 198 (e.g., a short-range communication network, such as Bluetooth™, wireless-fidelity (WiFi) direct, or infrared data association (IrDA)) or the second network 199 (e.g., a long-range communication network, such as a legacy cellular network, 5G network, next generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network and a next-generation communication technology, for example, a new radio (NR) access technology after a fourth generation (4G) network. The NR access technology may support high-speed transmission for high capacity data (enhanced mobile broadband; eMBB), terminal power minimizing and multiple terminal access (massive machine type communication; mMTC), or ultra-reliable and low-latency communications (URLLC). The wireless communication module 192 may support a high-frequency band (e.g., millimeter wave (mmWave) band) to achieve, for example, a higher data rate. The wireless communication module 192 may support various technologies, for example, beamforming, massive multiple-input and multiple-output (MIMO), Full-dimensional MIMO, an array antenna, analog beam-forming, or a large-scale antenna, to secure performance in high frequency bands. The wireless communication module 192 may support various requirements defined in the electronic device 101, the external electronic device (e.g., the electronic device 104) or the network system (e.g., the second network 199). According to one embodiment, the wireless communication module 192 may support a peak data rate (e.g., 20 Gbps or more) for eMBB realization, loss coverage (e.g., 164 dB or less) for mMTC realization, or user plane (U-plane) latency (e.g., 0.5 ms or less, or the round trip of 1 ms or less in each of a downlink (DL) and an uplink (UL)) for URLCC realization.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., printed circuit board (PCB)). According to an embodiment, the antenna module 197 may include a plurality of antennas (e.g., an array antenna). In such a case, at least one antenna appropriate for a communication scheme used in the communication network, such as the first network 198 or the second network 199, may be selected, for example, by the communication module 190 from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to an embodiment, the antenna module 197 may form an mmWave antenna module. According to an embodiment, the mmWave antenna module may include a printed circuit board, an RFIC disposed on a first surface (e.g., a bottom surface) of the printed circuit board, or disposed adjacent to the first surface to support the specific high frequency band (e.g., mmWave band), and a plurality of antennas (e.g., an array antenna) disposed on a second surface (e.g., a top surface or a side surface) of the printed circuit board or disposed adjacent to the second surface to transmit or receive a signal having the specified high frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, the command or data may be transmitted or received between the electronic device 101 and the external electronic device 104 through the server 108 connected to the second network 199. Each of the external electronic device 102 or 104 may be a device of which the type is the same as or different from that of the electronic device 101. According to an embodiment, all or a part of operations to be executed by the electronic device 101 may be executed in one or more external electronic devices among the external electronic devices 102, 104, or 108. For example, when the electronic device 101 needs to perform any function or service automatically or in response to a request from the user or any other device, the electronic device 101 may additionally request one or more external electronic devices to perform at least part of the function or service, instead of internally executing the function or service. The one or more external electronic devices which receive the request may execute at least a part of the function or service thus requested or an additional function or service associated with the request, and may provide a result of the execution to the electronic device 101. The electronic device 101 may process the result as it is or additionally, and may provide a result of the processing as at least a part of the response to the request. To this end, for example, cloud computing, distributed computing, mobile edge computing (MEC), or client-server computing may be used. For example, the electronic device 101 may provide an ultra-low latency service by using distributed computing or mobile edge computing. In another embodiment, the external electronic device 104 may include an Internet of Things (IoT) device. The server 108 may be an intelligent server using machine learning and/or a neural network. According to an embodiment, the external electronic device 104 or the server 108 may be included in the second network 199. The electronic device 101 may be applied to an intelligent service (e.g., a smart home, a smart city, a smart car, or a healthcare) based on fifth generation (5G) communication technology and IoT-related technology.

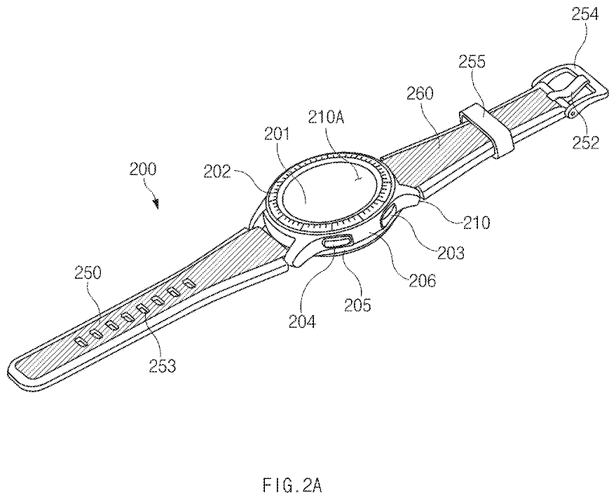

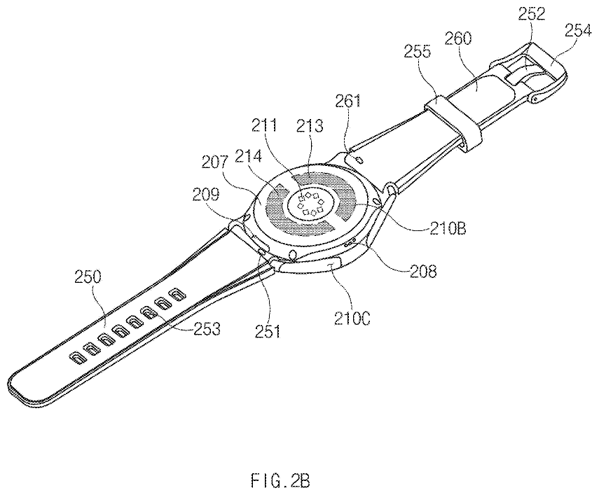

FIG. 2A is a perspective view of a front surface of an electronic device according to an embodiment of the disclosure.

FIG. 2B is a perspective view of a rear surface of the electronic device of FIG. 2A according to an embodiment of the disclosure.

Referring to FIGS. 2A and 2B, an electronic device 200 according to an embodiment (e.g., the electronic device 101 of FIG. 1) may include a housing 210 including a first surface (or a front surface) 210A, a second surface (or a rear surface) 210B, and a side surface 210C surrounding a space between the first surface 210A and the second surface 210B, and fastening members 250 and 260 connected to at least part of the housing 210 and configured to detachably fasten the electronic device 200 to a body part (e.g., a wrist or an ankle) of a user. In another embodiment (not illustrated), the housing may refer to a structure that forms a part of the first surface 210A, the second surface 210B, and side surfaces 210C of FIG. 2A. According to an embodiment, the first surface 210A may be formed by a front plate 201 (e.g., a glass plate including various coating layers, or a polymer plate), at least part of which is substantially transparent. The second surface 210B may be defined by a rear plate 207 that is substantially opaque. The rear plate 207, for example, may be formed by coated or colored glass, ceramic, a polymer, a metal (e.g., aluminum, stainless steel (STS), or magnesium), or a combination of at least two of the materials. The side surfaces 210C may be coupled to the front plate 201 and the rear plate 207, and may be defined by side members (or side bezel structures) 206 that include metal and/or polymer. In some embodiments, the rear plate 207 and the side members 206 may be integrally formed and may include the same material (e.g., a metal material such as aluminum). The fastening members 250 and 260 may be formed in various materials and shapes. The fastening members 250 and 260 may be formed such that the integral type and a plurality of unit links are capable of being moved with each other by woven fabric, leather, rubber, urethane, metal, ceramic, or the combination of at least two of the materials.

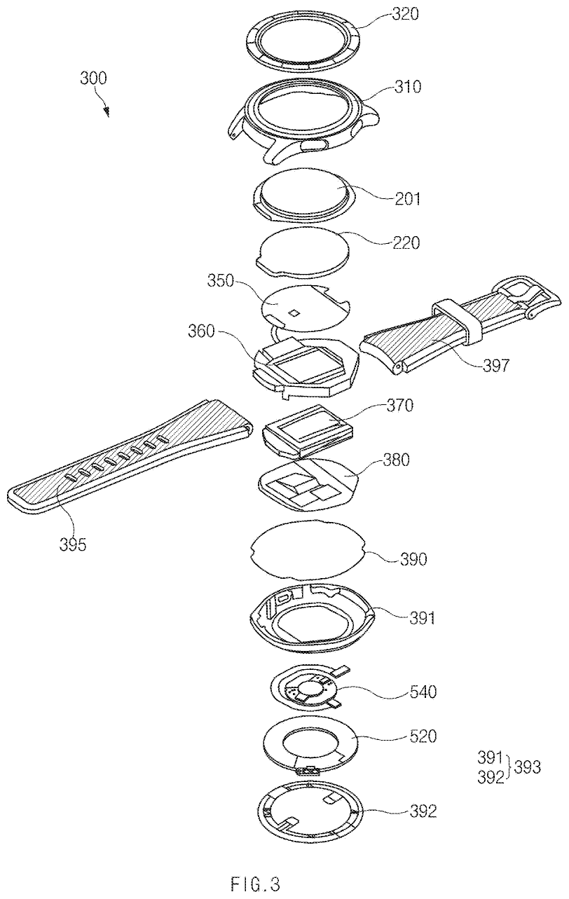

According to an embodiment, the electronic device 200 may include at least one of a display 220 (see FIG. 3), audio devices 205 and 208 (e.g., an audio module 170 of FIG. 1), a sensor 211 (e.g., a sensor module 176 of FIG. 1) (or a sensor assembly), key input devices 202, 203, and 204, and a connector hole 209. In some embodiments, the electronic device 200 may not include at least one (e.g., the key input devices 202, 203, and 204, the connector hole 209, or the sensor 211) of the components, or may further include any other component.

The display 220 may be exposed through a substantial portion of the front plate 201, for example. The display 220 may have a shape that corresponds to a shape of the front plate 201, and may have various shapes, such as a circular shape, an elliptical shape, or a polygonal shape. The display 220 may be coupled to a touch sensing circuit, a pressure sensor capable of measuring the intensity (or pressure) of a touch, and/or a fingerprint sensor or may be disposed adjacent thereto.

The audio devices 205 and 208 may include a microphone hole 205 and a speaker hole 208. A microphone for obtaining external sound may be disposed in the microphone hole 205, and in some embodiments, a plurality of microphones may be disposed to detect a direction of sound. The speaker hole 208 may be used as an external speaker and a call receiver. In some embodiments, the speaker hole 208 and the microphone hole 205 may be implemented as a single hole, or a speaker (e.g., a piezo speaker) may be included without the speaker hole 208.

The sensor 211 may generate an electrical signal or a data value that corresponds to an internal operation state of the electronic device 200 or an external environment state. The sensor 211 may be disposed on the second surface 210B of the housing 210, for example. The electronic device 200 may further include a sensor not illustrated, for example, at least one of a gesture sensor, a gyro sensor, a barometric pressure sensor, a magnetic sensor, an acceleration sensor, a grip sensor, a color sensor, an infrared (IR) sensor, a biometric sensor, a temperature sensor, a humidity sensor, or an illumination sensor.

The sensor 211 may include electrode areas 213 and 214 that form a portion of a surface of the electronic device 200, and a biosignal detecting circuit (not illustrated) that is electrically connected to the electrode areas 213 and 214. For example, the electrode areas 213 and 214 may include a first electrode area 213 and a second electrode area 214 that are disposed on the second surface 210B of the housing 210. The sensor 211 may be configured such that the electrode areas 213 and 214 acquire an electrical signal from a portion of the body of the user and a biosignal detecting circuit detects biometric information of the user based on the electrical signal.

The key input devices 202, 203, and 204 may include a wheel key 202 that is disposed on the first surface 210A of the housing 210 and is rotatable in at least one direction, and/or side key buttons 203 and 204 that are disposed on the side surface 210C of the housing 210. The wheel key 202 may have a shape that corresponds to a shape of the front plate 201. In another embodiment, the electronic device 200 may not include all or a part of the key input devices 202, 203, and 204 mentioned above, and the key input devices 202, 203, and 204 not included may be implemented on the display 220 in another form, such as a soft key. The connector hole 209 may include another connector hole (not illustrated) capable of accommodating a connector (e.g., a USB connector) for transmitting/receiving power and/or data to/from an external electronic device and accommodating a connector for transmitting/receiving an audio signal to/from the external electronic device. For example, the electronic device 200 may further include a connector cover (not illustrated) that covers at least part of the connector hole 209 and blocks an inflow of external foreign substances to the connector hole 209. In another embodiment, the electronic device 200 may not include some or all of the connector hole 209 and the connector cover.

The fastening members 250 and 260 may be detachably fastened to at least a partial area of the housing 210 by using locking members 251 and 261. The fastening members 250 and 260 may include at least one of a fixing member 252, a fixing member fastening hole 253, a band guide member 254, and a band fixing ring 255.

The fixing member 252 may be configured to fix the housing 210 and the fastening members 250 and 260 to a portion (e.g., a wrist or an ankle) of the body of the user. The fixing member fastening hole 253 may fix the housing 210 and the fastening members 250 and 260 to a portion of the body of the user in correspondence to the fixing member 252. The band guide member 254 may be configured to restrict a motion range of the fixing member 252 when the fixing member 252 and the fixing member fastening hole 253 are fastened to each other, so that the fastening members 250 and 260 may be closely attached and fastened to a portion of the body of the user. The band fixing ring 255 may restrict a motion range of the fastening members 250 and 260 in a state in which the fixing member 252 and the fixing member fastening hole 253 are fastened to each other. In another embodiment, the fastening members 250 and 260 may not include at least one of the fixing member 252, the fixing member fastening hole 253, the band guide member 254, and the band fixing ring 255. For example, because the fastening members 250 and 260 may be coupled to each other and formed in a ring shape, they may not include the fixing member 252, the fixing member fastening hole 253, the band guide member 254, and/or the band fixing ring 255.

FIG. 3 is an exploded perspective view illustrating an electronic device according to an embodiment of the disclosure.

Referring to FIG. 3, the electronic device 300 (e.g., the electronic device 101 of FIG. 1 or the electronic device 200 of FIGS. 2A and 2B) may include a side member 310 (e.g., the side member 206 of FIG. 2A) (or a side frame, side member), a wheel key 320 (e.g., the wheel key 202 of FIG. 2A), a front plate 201, a display 220, an antenna 350, a support member 360 (e.g., a bracket), a battery 370, a printed circuit board 380 (or a first circuit board, a main circuit board), a sealing member 390, a rear plate 393 (e.g., the rear plate 207 of FIG. 2B), and fastening members 395 and 397 (e.g., the fastening members 250 and 260 of FIGS. 2A and 2B). At least one of the components of the electronic device 300 may be similar to or the same as at least one of the components of the electronic device (200 of FIG. 1 or 2), and thus, repeated descriptions thereof will be omitted. The support member 360 may be disposed in the electronic device 300 and may be connected to the side member 310, or may be integrally formed with the side member 310. For example, the support member 360 may be formed of a metal material and/or a nonmetal material (e.g., polymer). The display 220 may be coupled to one surface of the support member 360, and the printed circuit board 380 may be coupled to an opposite surface of the support member 360. A processor, a memory, and/or an interface may be mounted on the printed circuit board 380. The processor may include, for example, one or more of a central processing unit, a graphic processing device, a sensor processor, or a communication processor.

The memory may include, for example, a volatile memory or a nonvolatile memory. The interface may include, for example, a high definition multimedia interface (HDMI), a universal serial bus (USB) interface, a secure digital (SD) card interface, and/or an audio interface. The interface may electrically or physically connect, for example, the electronic device 300 to an external electronic device and may include a USB connector, an SD card/MMC connector, or an audio connector.

The battery 370 that is a device for supplying power to at least one component of the electronic device 300 may include, for example, a primary cell incapable of being recharged, a secondary cell rechargeable, or a fuel cell. At least part of the battery 370 may be disposed on substantially the same plane as the printed circuit board 380, for example. The battery 370 may be integrally disposed in an interior of the electronic device 200, or may be disposed in the electronic device 200 to be detachable.

The sealing member 390 may be located between the side member 310 and the rear plate 393. The sealing member 390 may be configured to block moisture and foreign substances that are introduced into a space surrounded by the side member 310 and the rear plate 393 from an outside.

The rear plate 393 may include a first cover plate 391 that is coupled to the side member 310 and a second cover plate 392 that is coupled to the first cover plate 391.

The second cover plate 392 may be formed to be at least partially transparent so that light may pass therethrough for an operation of a biometric sensor unit 540. For example, the second cover plate 392 may include a transparent area that is formed at a position corresponding to a light emitting unit and a light receiving unit of the biometric sensor unit 540. The light generated in the light emitting unit of the biometric sensor unit 540 may pass through the transparent area of the second cover plate 392 and reach an external object (e.g., a wrist of the user), and light reflected from the external object may pass through the transparent area of the second cover plate 392 and reach the light receiving unit.

The rear plate 393 may support a wireless charging part 520 and a biometric sensor unit 540. For example, the wireless charging part 520 and the biometric sensor unit 540 may be disposed in a space between the first cover plate 391 and the second cover plate 392. The wireless charging part 520 may generate an induced current in response to an external electromagnetic field, and may supply power to the electronic device 300 or charge the battery 370 by using the induced current. A coil antenna included in the wireless charging part 520 may, for example, perform short-range communication with an external device or may wirelessly transmit and receive power necessary for charging, and may transmit a short-range communication signal or a magnetic-based signal that includes payment data. In another embodiment, an antenna structure may be formed by a portion of the side member 310 and/or a portion of the rear plate 393, or by a combination thereof.

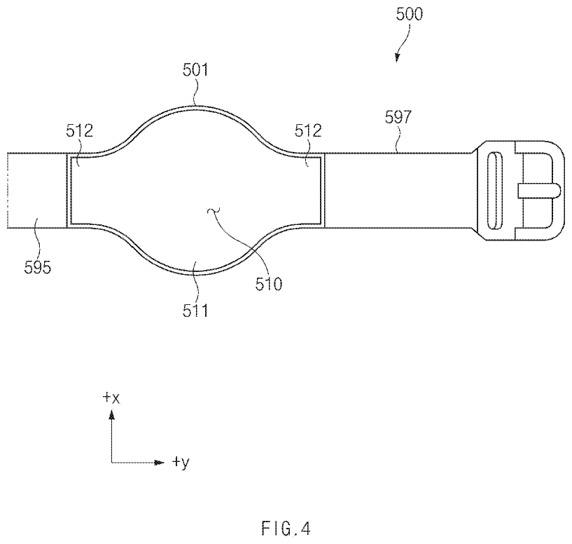

FIG. 4 is a view illustrating a portion of a wearable electronic device according to an embodiment of the disclosure.

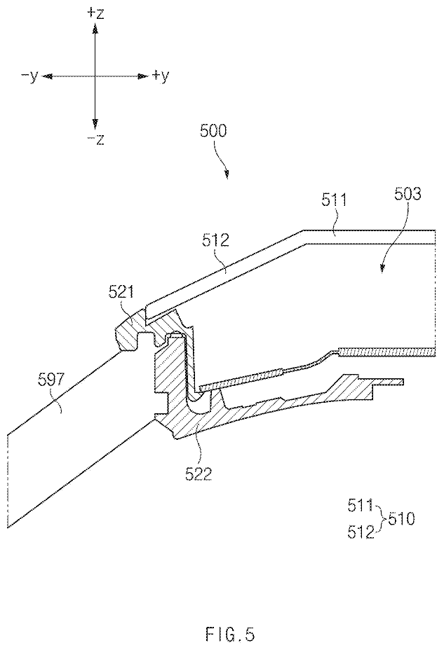

FIG. 5 is a view illustrating a housing and fastening members of a wearable electronic device according to an embodiment of the disclosure.

A wearable electronic device 500 of FIGS. 4 and 5 may be at least partially similar to at least one of the electronic device 101 of FIG. 1, the electronic device 200 of FIGS. 2A and 2B, and the electronic device 300 of FIG. 3, or may include other embodiments of the electronic device.

Referring to FIGS. 4 and 5, the wearable electronic device 500 may include a housing 501 and fastening members 595 and 597.

The housing 501 may be formed of a conductive member (e.g., a metal) or a non-conductive member (e.g., PC, rubber, or urethane). According to an embodiment, in the housing 501, a conductive member may be formed by being insert-injected into at least a part of a non-conductive member.

The housing 501 may include a front plate 510 (e.g., the front plate 201 of FIGS. 2A and 2B) (or a first cover member), a rear plate 522 (e.g., the rear plate 207 of FIG. 2) (or a second cover member) that faces in a direction opposite to the front plate 510, and/or a side member 521 (e.g., the side member 206 of FIGS. 2A and 2B, the side member 310 of FIG. 3) that surrounds an inner space 503 between the front plate 510 and the rear plate 522.

The front plate 510 may be disposed on at least one of a display (e.g., the display module 160 of FIG. 1 or the display 220 of FIG. 3) and a sensor (e.g., the sensor module 176 of FIG. 1) (or at a position that faces the +z-axis direction). The front plate 510 may define at least a portion of an outer surface of the electronic device 500. For example, the front plate 510 may define at least a portion of a front surface (e.g., the first surface 210a and the second surface 220a of FIGS. 2A and 2B) of the electronic device 500. According to an embodiment, the front plate 510 may cover at least a portion of at least any one of the display and the sensor, and may protect at least any one of the display and the sensor from an external impact.

According to an embodiment, the front plate 510 may include a glass plate or a polymer plate that includes at least one coating layer. According to an embodiment, the front plate 510 may, for example, include a window glass (for example, a cover window, a cover glass, a glass window, or a transparent window) for protecting from an external impact.

According to an embodiment, the front plate 510 may be formed to be longer in the other direction than in any one direction, among a widthwise direction (or the +x-axis direction) and a lengthwise direction (or the +y-axis direction) of the pair of fastening members 595 and 597. For example, the front plate 510 may be formed to be longer in a lengthwise direction (or the +y-axis direction) of the pair of fastening members 595 and 597 than in a widthwise direction (or the +x-axis direction) of the pair of fastening members.

According to an embodiment, the front plate 510 may include at least one first window area 511 and at least one second window area 512. As an example, the second window area 512 may be disposed on one side or opposite sides of the first window area 511. The first window area 511 may correspond to the display. The first window area 511 may correspond to a size and/or a position of the display. The second window area 512 may correspond to the sensor. The second window area 512 may correspond to a size and/or a position of the sensor.

According to an embodiment, the second window area 512 may be disposed between the side member 521 and the first window area 511. The second window area 512 may extend from the first window area 511 toward the side member 521 and/or the pair of fastening members 595 and 597. At least a portion of the second window area 512 may be formed to be inclined. The second window area 512 may be disposed to be inclined downward toward the side member 521 from the first window area 511. As an example, at least a portion of the second window area 512 may be disposed to be inclined downward along the-z-axis direction from a periphery of the first window area 511 toward the-y-axis direction, with respect to a cross section that is parallel to the −z-axis direction and the +z-axis direction. The second window area 512 may be disposed to be inclined upward toward the first window area 511 from the side member 521. As an example, at least a portion of the second window area 512 may be disposed to be inclined upward along the +z-axis direction from a periphery of the side member 521 toward the +y-axis direction, with respect to a cross section that is parallel to the-z-axis direction and the +z-axis direction. The second window area 512 may be formed to form an obtuse angle with the first window area 511.

According to an embodiment, the first window area 511 and the second window area 512 may be formed of different materials or may be formed of a same material. For example, the first window area 511 and the second window area 512 may be formed of a same material to be integrated.

According to an embodiment, at least any one of the first window area 511 and the second window area 512 included in the front plate 510 may be formed of a transparent and flexible material. For example, at least any one of the first window area 511 and the second window area 512 may be formed of glass.

According to an embodiment, the rear plate 522 of the electronic device 500 may contact a wrist of the user, and may at least partially include a conductive member (e.g., the electrode areas 213 and 214 of FIG. 2B) to acquire biometric information of the user.

According to an embodiment, the pair of fastening members 595 and 597 (e.g., a connection part or a strap) (e.g., the fastening members 395 and 397 of FIGS. 2A and 2B) may be disposed at an outside of the housing 501. The fastening members 595 and 597 may include a first fastening member 595 that is connected to one side of the housing 501, and a second fastening member 597 that is connected to an opposite side of the housing 501. The electronic device 500 may be mounted by winding the fastening members 595 and 597 on a wrist while it is positioned on the wrist of the user.

According to an embodiment, at least any one of the first fastening member 595 and the second fastening member 597 may be connected to at least any one of the side member 521 and the rear plate 522. A portion of at least any one of the first fastening member 595 and the second fastening member 597 may be connected to the side member 521, and a remaining portion of at least any one of the first fastening member 595 and the second fastening member 597 may be connected to the rear plate 522.

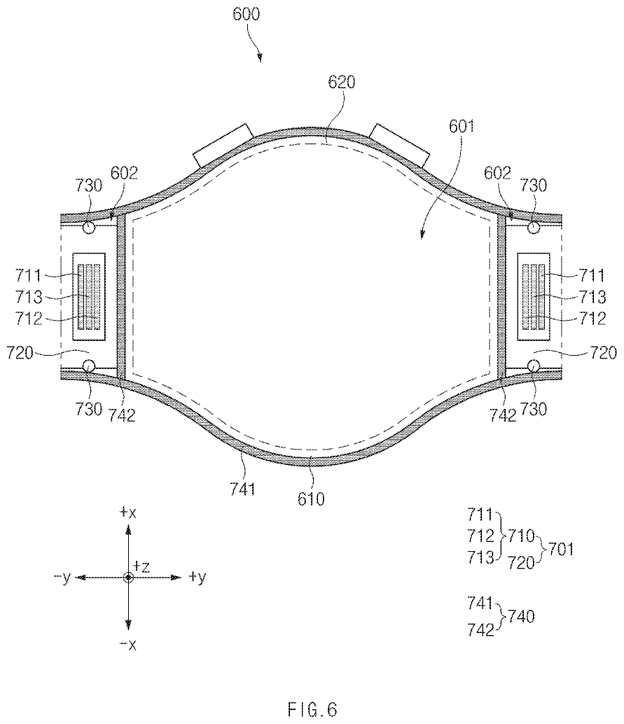

FIG. 6 is a view illustrating a portion of a wearable electronic device 600 according to an embodiment of the disclosure.

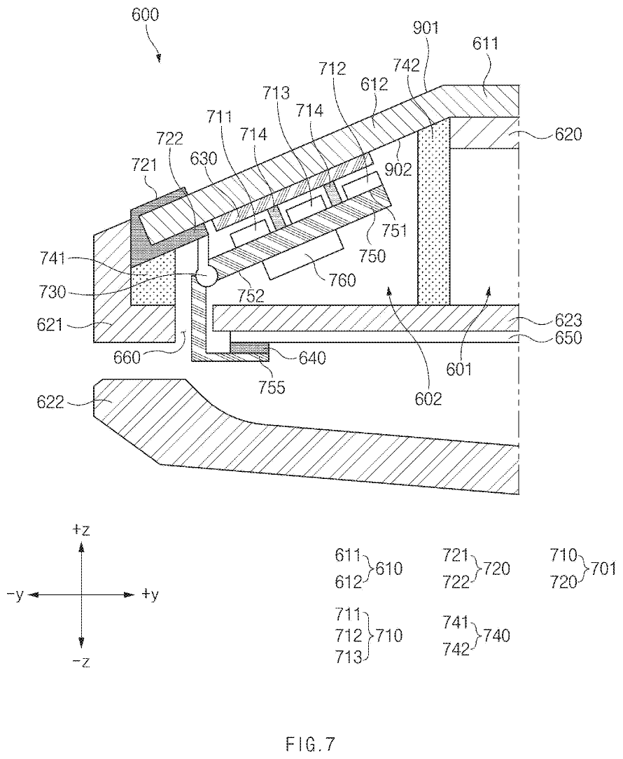

FIG. 7 is a view illustrating a cross section of the sensor assembly 701 illustrated in FIG. 6, taken along the +y-axis direction and the −y-axis direction according to an embodiment of the disclosure.

The wearable electronic device 600 of FIGS. 6 and 7 may be at least partially similar to at least one of the electronic device 101 of FIG. 1, the electronic device 200 of FIGS. 2A and 2B, the electronic device 300 of FIG. 3, and the electronic device 500 of FIGS. 4 and 5, or may include other embodiments of the electronic device.

Referring to FIGS. 6 and 7, a wearable electronic device 600 according to an embodiment may include a side member 621 (e.g., the side member 521 of FIG. 5), a front plate 610 (e.g., the front plate 510 of FIGS. 4 and 5), a molding member 740, a display 620 (e.g., the display module 160 of FIG. 1), and a sensor assembly 701 (or a sensor) (e.g., the sensor module 176 of FIG. 1).

The display 620 may be disposed between the front plate 610 and the bracket 623. The display 620 may be visually exposed in a front direction (the +z-axis direction) of the electronic device 600 through the front plate 610. The display 620 may be visible in a forward direction (the +z-axis direction) of the electronic device 600 through the front plate 610. The display 620 may be attached to a rear surface (a surface that faces the −z-axis direction) of the front plate 610. The display 620 may be disposed to face the printed circuit board 650 with the bracket 623 interposed therebetween, and the display 620 may be electrically connected to the printed circuit board 650.

The front plate 610 may include at least one first window area 611 (e.g., the first window area 511 of FIGS. 4 and 5) and at least one second window area 612 (e.g., the second window area 512 of FIGS. 4 and 5).

The first window area 611 may include one surface that faces the +z-axis direction and an opposite surface that faces the −z-axis direction. The opposite surface of the first window area 611 may face the display 620 (for example, an active area of the display 620, or a display area) that is accommodated in the first inner space 601. The first window area 611 may be disposed to overlap the display 620 with a size that corresponds to the display 620.

The second window area 612 may be formed to extend from one side and/or an opposite side of the first window area 611 in the −y-axis direction (and/or the −x-axis direction) and/or the +y-axis direction (and/or the +x-axis direction). The second window area 612 may be formed to extend in the −y-axis direction and the +y-axis direction with respect to the first window area 611. The second window area 612 may be formed to extend in a direction (for example, the y-axis direction and/or the −y-axis direction) that is perpendicular to a first direction (the +z-axis direction) which the front plate 610 faces and a second direction (the −z-axis direction) which the rear plate 622 faces.

The second window area 612 may include one surface 901 that faces a diagonal direction between each of the +y-axis direction and the −y-axis direction, and the +z-axis direction, and may include an opposite surface 902 that faces a diagonal direction between each of the +y-axis direction and the −y-axis direction, and the −z-axis direction. The opposite surface 902 of the second window area 612 may face the sensor assembly 701 that is accommodated in the second inner space 602.

The one surface 901 of the second window area 612 may be configured to contact a portion (e.g., a finger of the user) of an external object (or the user) that wears the wearable electronic device 600.

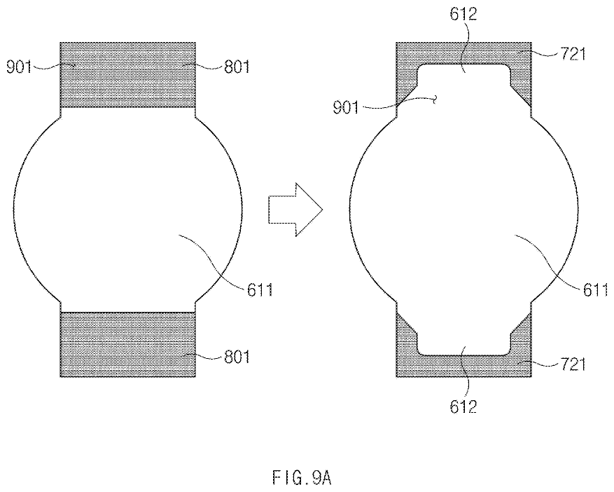

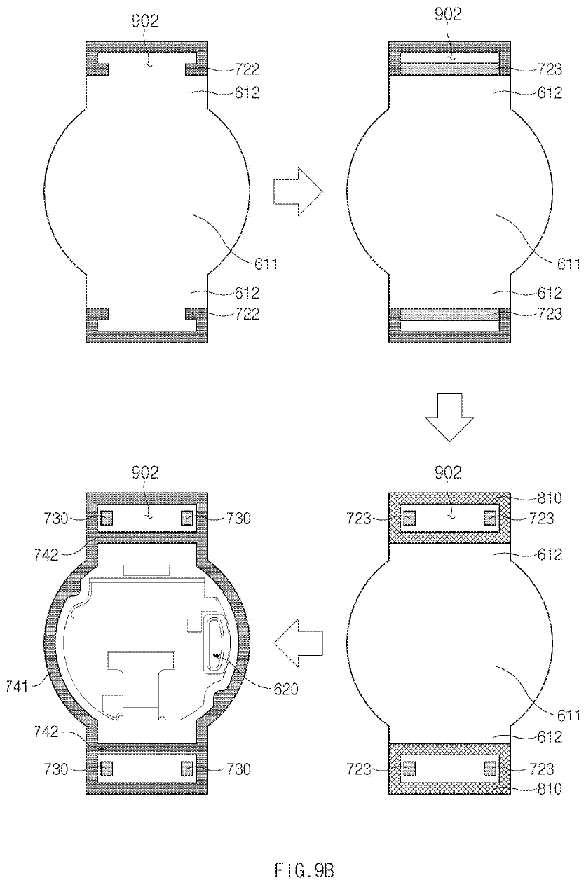

According to an embodiment, the molding member 740 may be disposed between the front plate 610 and the rear plate 622. The molding member 740 may divide (or partition) an inner space (e.g., the inner space 503 of FIG. 5) between the front plate 610 and the rear plate 622 into a first inner space 601 and a second inner space 602. The molding member 740 may include a first molding member 741 that is formed parallel to a portion of the side member 621, and, referring to FIG. 9B, a second molding member 742 that is connected to the first molding member 741 and is formed parallel to a portion of the side member 621.

At least a portion of the first molding member 741 may be disposed between the front plate 610 and a portion of the side member 621, when viewed in the +y-axis direction and/or the −y-axis direction. At least a portion of the first molding member 741 may be disposed on an inner side of the side member 621 along the side member 621. The first molding member 741 may be formed to surround at least a portion of a periphery of the first inner space 601 in which the display 620 is accommodated and/or at least a portion of a periphery of the second inner space 602 in which the sensor assembly 701 is accommodated. The first molding member 741 may be formed along a portion of a circumference of the display 620 and a portion of a circumference of the sensor assembly 701.

The second molding member 742 may be disposed between the front plate 610 and the bracket 623, when viewed in the +y-axis direction and/or the −y-axis direction. The second molding member 742 may be disposed between the first inner space 601 and the second inner space 602. Because the second molding member 742 absorbs or blocks light generated from the display 620 that is disposed in the first inner space 601, the second molding member 742 may minimize and/or prevent the light generated from the display 620 from being introduced into the second inner space 602.

According to an embodiment, at least any one of the first molding member 741 and the second molding member 742 may be formed of an opaque material. At least any one of the first molding member 741 and the second molding member 742 may be formed of an insulating material having elasticity (e.g., a silicone material). At least any one of the first molding member 741 and the second molding member 742 may be formed of an insulating material that is opaque and has elasticity.

According to an embodiment, a portion of the first molding member 741 and/or at least a portion of the second molding member 742 may be formed at an outer periphery of the display 620. When the display 620 is viewed from above, a portion of the first molding member 741 and/or at least a portion of the second molding member 742 may be formed to overlap a dead space DS (or a bezel, a non-display area) of the display 620.

According to an embodiment, the sensor assembly 701 may include at least one of a heartbeat rate monitor (HRM) sensor that measures a heart rate of a user, an electrocardiogram (ECG) sensor that measures an electrocardiogram of the user, a photoplethysmography (PPG) sensor that measures a photoplethysmography of the user, a bioelectric impedance analysis (BIA) sensor that measures a body fat percentage of the user, and/or a galvanic skin response (GSR) sensor that measures a skin resistance of the user. As an example, among a plurality of sensors, the heartbeat rate monitor (HRM) sensor may be a sensor that irradiates light onto a body of the user (e.g., a finger of the user) and, by using a characteristic reflected from the finger of the user, estimates various biometric states based on a movement of blood vessels distributed in the finger of the user. The sensor assembly 701 may selectively include not only a sensor for measuring a biometric signal but also other sensors as needed (e.g., an angular velocity sensor, an acceleration sensor, or a wear detection sensor), and types thereof may not be limited thereto.

According to an embodiment, the sensor assembly 701 may include a first biometric sensor unit 710 (or an optical measurement sensing unit) and a second biometric sensor unit 720 (or an electrical measurement sensing unit).

The first biometric sensor unit 710 may be disposed on a flexible printed circuit board (FPCB) 750. The flexible printed circuit board 750 may include one surface 751 that faces the second window area 612 of the front plate 610 and an opposite surface 752 that faces an opposite direction to the one surface 751. At least a portion of at least any one of the one surface 751 and the opposite surface 752 of the flexible printed circuit board 750 may be formed parallel to at least any one of the one surface 901 of the second window area 612 and the opposite surface 902 of the second window area 612. According to an embodiment, the flexible printed circuit board 750 may include an extension part 755 that extends toward the printed circuit board 650. The extension part 755 may be disposed in a space between the rear plate 622 and an opposite surface (e.g., a surface that faces the −z-axis direction) of the printed circuit board 650 via a hole 660. As an example, the hole 660 may be formed to pass through one surface (e.g., a surface that faces the +z-axis direction) and an opposite surface (e.g., a surface that faces the-z-axis direction) of at least any one of the side member 621 and the bracket 623. As an example, the hole 660 may be a spaced space between the side member 621 and the bracket 623. The extension part 755 of the flexible printed circuit board 750 may be electrically connected to the printed circuit board 650 through the connector 640. According to an embodiment, at least a portion of at least any one of the flexible printed circuit board 750 and an optical film 630 may be disposed parallel to the second window area 612. At least a portion of at least any one of the flexible printed circuit board 750 and the optical film 630 may be disposed to have the same inclination angle as the second window area 612. According to an embodiment, at least a portion of at least any one of the printed circuit board 650 and the bracket 623 may be disposed parallel to the first window area 611.

According to an embodiment, the first biometric sensor unit 710 may optically measure a finger portion in which blood vessels are located near the skin. A distance between a skin surface of a finger and blood vessels may be smaller than a distance between a skin surface of a wrist and blood vessels. The first biometric sensor unit 710 may use various wavelength bands of light, and may acquire biometric information of the user even with emission of a small amount of light. As an example, the first biometric sensor unit 710 may precisely measure substances distributed in blood vessels at various wavelengths by measuring a movement of blood vessels distributed in a finger.

According to an embodiment, the first biometric sensor unit 710 may measure an amount of an antioxidant substance accumulated in skin and/or may measure an amount of a glycated protein accumulated in the skin, which is required for blood glucose management.

According to an embodiment, the first biometric sensor unit 710 may include at least one light emitting unit 711 and 712 and at least one light receiving unit 713. Light of a specific wavelength band output from the light emitting units 711 and 712 may be irradiated onto the body of a user. The light emitting units 711 and 712 may irradiate light having a predetermined intensity onto the body of a user, and a wavelength of the irradiated light may vary depending on a measurement purpose or a type of a target component to be analyzed. For example, the light emitting units 711 and 712 may include at least one light emitting body including a light emitting diode (LED) or a laser diode (LD). The light emitting units 711 and 712 may use various wavelength bands, such as green light for reducing an influence of motion artifact, red light, blue light, yellow light, ultraviolet (UV) light, or infrared light. The light emitting units 711 and 712 may emit light by turning on multiple wavelengths sequentially or simultaneously, or in an alternating manner. The light receiving unit 713 may receive at least a portion of reflected light that is irradiated and returned, and may generate at least one piece of biometric information by using an electrical signal obtained by converting the light. For example, the light receiving unit 713 may include a photodiode (PD), a photo transistor, or a charge-coupled device (CCD). The light receiving unit 713 may be any element that converts an optical signal into an electrical signal, and a type of the element may not be limited thereto.

According to an embodiment, the first biometric sensor unit 710 may include a first light emitting unit 711, a second light emitting unit 712, and a light receiving unit 713. The first light emitting unit 711 and the second light emitting unit 712 may emit light of the same wavelength band or may emit light of different wavelength bands. As an example, the first light emitting unit 711 and the second light emitting unit 712 included in the first biometric sensor unit 710 disposed on a left side (e.g., a side in the-y-axis direction) of the display 620 may emit light of a different wavelength band from that of the first light emitting unit 711 and the second light emitting unit 712 included in the first biometric sensor unit 710 disposed on a right side (e.g., a side in the +y-axis direction) of the display 620. The light receiving unit 713 may receive reflected light that is generated from at least any one of the first light emitting unit 711 and the second light emitting unit 712 and is reflected from an external object (e.g., a body of a user).

At least any one of the first light emitting unit 711 and the second light emitting unit 712 may generate light and may irradiate the light onto a finger of a user that is in contact with the second window area 612 of the front plate 610. Light emitted by at least any one of the first light emitting unit 711 and the second light emitting unit 712 may be reflected by a finger of a user that is in contact with the second window area 612 of the front plate 610. Light reflected by a finger of a user may reach the light receiving unit 713 of the first biometric sensor unit 710. The first biometric sensor unit 710 may acquire biometric information of a user based on light received through the light receiving unit 713.

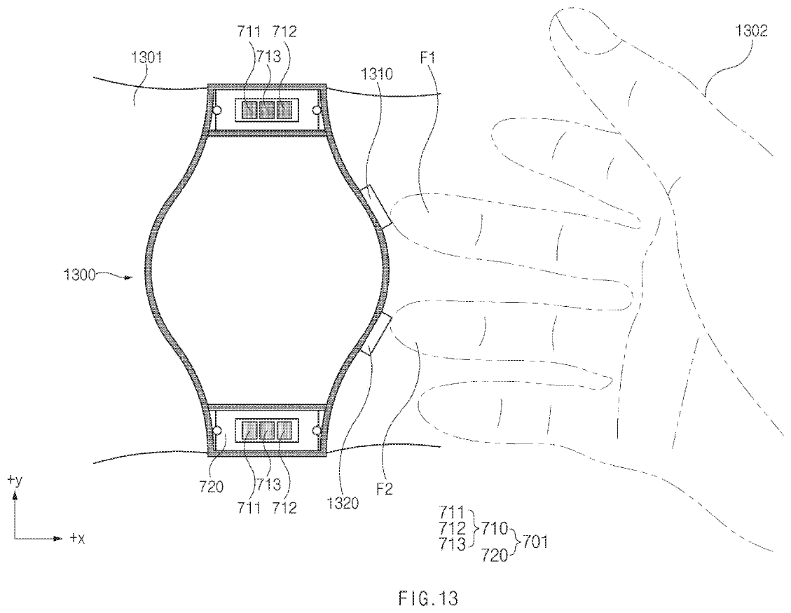

According to an embodiment, at least a portion of the light receiving unit 713 may be disposed to be surrounded by a plurality of light emitting units 711 and 712. The light receiving unit 713 may be disposed between the first light emitting unit 711 and the second light emitting unit 712. As an example, as illustrated in FIG. 13, the light receiving unit 713 may be disposed between the first light emitting unit 711 and the second light emitting unit 712 that are arranged in a line along the +x-axis direction or the −x-axis direction. As an example, as illustrated in FIG. 6, the light receiving unit 713 may be disposed between the first light emitting unit 711 and the second light emitting unit 712 that are arranged in a line along the +y-axis direction or the −y-axis direction.

According to an embodiment, the wearable electronic device 600 may include a partition wall 714. The partition wall 714 may extend from the one surface 751 of the flexible printed circuit board 750 toward the front plate 610 and/or the optical film 630. The partition wall 714 may be disposed between the light emitting units 711 and 712 and the light receiving unit 713 included in the first biometric sensor unit 710, and/or may be disposed between the light emitting units 711 and 712, and/or may be disposed between the light receiving units 713.

The partition wall 714 may prevent light emitted from at least one light emitting unit 711 and 712 from being directly incident on the light receiving unit 713 without passing through an external object. The partition wall 714 may be formed of a material that absorbs light to prevent light of any wavelength band from being directly incident on the light receiving unit 713.

The partition wall 714 may be disposed between the flexible printed circuit board 750 and the optical film 630. The partition wall 714 may be fixed to the flexible printed circuit board 750 in a form of a surface mount device (SMD), and may be attached to the optical film 630 through an adhesive tape (not illustrated).

According to an embodiment, the wearable electronic device 600 may include an optical film 630. The optical film 630 may be attached to the opposite surface 902 of the second window area 612. The optical film 630 may be disposed in an area that corresponds to the first biometric sensor unit 710. The optical film 630 may control an optical path by applying a pattern to a polymer film (e.g., polyethylene terephthalate (PET)). The optical film 630 may control an optical path of light emitted from at least any one of the first light emitting unit 711 and the second light emitting unit 712 and/or an optical path of light that is reflected by an external object and returns to the light receiving unit 713. The optical film 630 may control light emitted from at least any one of the first light emitting unit 711 and the second light emitting unit 712 such that the light passes through the front plate 610 and is emitted to an external object. The optical film 630 may control light reflected by an external object such that the light passes through the front plate 610 and is incident on the light receiving unit 713.

According to an embodiment, while the wearable electronic device 600 is worn, a heart rate of a user may be measured by contacting the second biometric sensor unit 720 with a finger of the user.

According to an embodiment, the second biometric sensor unit 720 may include a first conductive member 721 (or an external electrode) and a second conductive member 722 (or an internal electrode). The first conductive member 721 may be formed to define a portion of a front surface of the housing (e.g., the housing 501 of FIG. 4). The first conductive member 721 may contact a portion (e.g., a finger) of the body of a user. The first conductive member 721 and the second conductive member 722 may be electrically connected to each other. The second conductive member 722 may be electrically connected to a circuit area (not illustrated) of the flexible printed circuit board 750 through the conductive connection member 730.

According to an embodiment, at least any one of the first conductive member 721 and the second conductive member 722 included in the second biometric sensor unit 720 may be formed of a conductive material in an area excluding an optical path of the first biometric sensor unit 710 included in the sensor assembly 701. At least any one of the first conductive member 721 and the second conductive member 722 included in the second biometric sensor unit 720 may be disposed in consideration of a position of the first biometric sensor unit 710. At least any one of the first conductive member 721 and the second conductive member 722 may be formed at at least any one of an upper side (e.g., one side in the +x-axis direction), a lower side (e.g., one side in the −x-axis direction), a left side (e.g., one side in the −y-axis direction), and a right side (e.g., one side in the +y-axis direction) of the first biometric sensor unit 710. As an example, at least any one of the first conductive member 721 and the second conductive member 722 included in the second biometric sensor unit 720 may be formed to surround the first biometric sensor unit 710.

According to an embodiment, at least a portion of the first conductive member 721 may be disposed outside the second inner space 602. At least a portion of the second conductive member 722 may be disposed inside the second inner space 602. As an example, at least any one of the first conductive member 721 and the second conductive member 722 may require a surface area of at least 25 mm² to minimize a surface resistance. At least any one of the first conductive member 721 and the second conductive member 722 may have a degree of design freedom in an up-down direction or a left-right direction together with the conductive connection member 730 in consideration of positions of the light emitting units 711 and 712 and the light receiving unit 713.

According to an embodiment, the first conductive member 721 may be disposed on the one surface 901 (e.g., a surface that faces the +z-axis direction) of the second window area 612 of the front plate 610 and on a side surface (e.g., a surface that faces the side member, a surface that faces the y-axis direction). The second conductive member 722 may be disposed on the opposite surface 902 (e.g., a surface that faces the -z-axis direction) of the second window area 612 of the front plate 610 and on a side surface (e.g., a surface that faces the side member, or a surface that faces the y-axis direction). The first conductive member 721 and the second conductive member 722 disposed on a side surface of the second window area 612 of the front plate 610 may directly contact each other on the side surface of the second window area 612.

According to an embodiment, any one of the first conductive member 721 and the second conductive member 722 may be disposed on a portion of a side surface of the second window area 612 of the front plate 610. As an example, the first conductive member 721 may be disposed on the one surface 901 and a side surface of the second window area 612 of the front plate 610, and the second conductive member 722 may be disposed on the opposite surface 902 of the second window area 612 of the front plate 610. The first conductive member 721 disposed on a side surface of the second window area 612 of the front plate 610 may contact the second conductive member 722 near a corner between the side surface of the second window area 612 and the opposite surface 902. As an example, the first conductive member 721 may be disposed on the one surface 901 of the second window area 612 of the front plate 610, and the second conductive member 722 may be disposed on the opposite surface 902 and a side surface of the second window area 612 of the front plate 610. The second conductive member 722 disposed on a side surface of the second window area 612 of the front plate 610 may contact the first conductive member 721 near a corner between the side surface and the one surface 901 of the second window area 612.

According to an embodiment, the first conductive member 721 may be disposed on the one surface 901 of the second window area 612 of the front plate 610. The second conductive member 722 may be disposed on the opposite surface 902 of the second window area 612 of the front plate 610. The first conductive member 721 and the second conductive member 722 disposed on different planes may be electrically connected through the side member 621 formed of a conductive material.

According to an embodiment, the wearable electronic device 600 may include a control circuit 760 (e.g., an analog front end integrated circuit (AFE IC)). The control circuit 760 may be disposed on the opposite surface 752 of the flexible printed circuit board 750. On the one surface 751 of the flexible printed circuit board 750, the conductive connection member 730 that contacts the second biometric sensor unit 720 and at least one light emitting unit 711 and 712 and at least one light receiving unit 713 included in the first biometric sensor unit 710 may be disposed. The control circuit 760 may preprocess an electrical analog signal output from the second biometric sensor unit 720 and/or the first biometric sensor unit 710. The control circuit 760 may include at least one converter (e.g., a first converter 117 and/or a second converter 118) (e.g., an analog digital converter (ADC)) that converts an electrical analog signal into a digital signal output from the second biometric sensor unit 720 and/or the first biometric sensor unit 710 based on power provided under the control of a processor (e.g., the processor 120 of FIG. 1). Furthermore, the control circuit 760 may further include an amplifier for amplifying an analog signal output from the second biometric sensor unit 720 and/or the first biometric sensor unit 710 and a digital signal output from at least one converter.

A digital signal converted in the control circuit 760 may be transmitted to a processor (e.g., the processor 120 of FIG. 1). The processor may be disposed on a printed circuit board 650. The control circuit 760 may transmit biometric information in a digital form to the processor by performing digital communication with the processor through an extension part 755 of a flexible printed circuit board 750, through which the flexible printed circuit board 750 passes through the hole 660. The processor may obtain user biometric information based on a digital signal received from the control circuit 760. For example, the processor may output the obtained biometric information to the display 620, store the obtained biometric information in a memory, or transmit the obtained biometric information to another electronic device or a server through a communication device (e.g., the communication module 190 of FIG. 1) and/or an antenna. When the biometric information satisfies a specified condition, the processor may provide a warning signal (e.g., a warning screen or sound) to a user through the display 620 or a speaker (not illustrated), or transmit the corresponding biometric information to a related institution (e.g., a hospital). For example, the specified condition may include a state in which there is an abnormality in a health of the user.

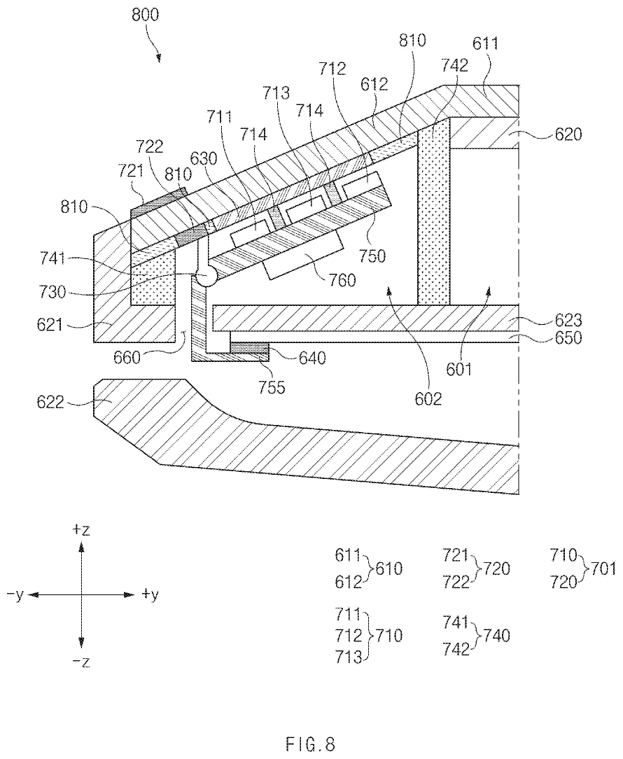

FIG. 8 is a view illustrating a cross section of a portion of a wearable electronic device 800 according to an embodiment of the disclosure.

Referring to FIG. 8, a wearable electronic device 800 according to an embodiment may include a side member 621 (e.g., the side member 521 of FIG. 5), a front plate 610 (e.g., the front plate 510 of FIGS. 4 and 5), a molding member 740, a display 620 (e.g., the display module 160 of FIG. 1), and a sensor assembly 701 (e.g., the sensor module 176 of FIG. 1). The electronic device illustrated in FIG. 8 may have a configuration that is substantially the same as that of the electronic device illustrated in FIG. 7, except that the electronic device illustrated in FIG. 8 further includes a protective member 810. Accordingly, the above descriptions may be applied to the same configurations or operations, and thus, a repeated description of configurations having the same reference numerals will be omitted.

According to an embodiment, the protective member 810 may be formed on an opposite surface (e.g., the opposite surface 902 of the second window area 612) of the front plate 610. The protective member 810 may be formed in a remaining area excluding an area in which the first biometric sensor unit 710, the second biometric sensor unit 720, and the display 620 are formed. The protective member 810 may be formed in a remaining area excluding an area in which at least any one of the display 620, the optical film 630, the second conductive member 722, the first molding member 741, and the second molding member 742 is formed. The protective member 810 may be formed on an opposite surface of the front plate 610 in a remaining area excluding an area in which the display 620, the optical film 630, the second conductive member 722, the first molding member 741, and the second molding member 742 are formed. As an example, the protective member 810 may be formed on an opposite surface of the front plate 610 between the second conductive member 722 and the side member 621, between the second conductive member 722 and the optical film 630, and between the second molding member 742 and the optical film 630.

According to an embodiment, the protective member 810 may provide an aesthetic appearance to the wearable electronic device 800 as ink of a specified color, which is used for forming an external design of the wearable electronic device 800, is printed on an opaque material or on the protective member 810.

According to an embodiment, the protective member 810 may be formed of a non-conductive (or insulating) material to prevent a short between the second biometric sensor unit 720 and other electrical components.

FIG. 9A is views illustrating a method for manufacturing a structure disposed on one surface of a front plate included in a wearable electronic device according to an embodiment of the disclosure.

Referring to FIG. 9A, a front plate that includes a first window area 611 and a second window area 612 (e.g., the front plate 510 of FIGS. 4 and 5, the front plate 610 of FIGS. 6 to 8) may be provided. A second conductive member (e.g., the second conductive member 722 of FIGS. 6, 7, and 8) may be formed on the front plate 610, or the second conductive member may not be formed on the front plate 610. After a first conductive material 801 is deposited on one surface 901 of the second window area 612, a first conductive member 721 may be formed on a specified portion of one surface 901 of the second window area 612 by etching the first conductive material 801 using a laser. For example, the first conductive material 801 may be formed in a single-layer or multi-layer structure that includes at least any one of Ti and Al.

FIG. 9B is views illustrating a method for manufacturing a structure disposed on an opposite surface of a front plate included in a wearable electronic device according to an embodiment of the disclosure.

Referring to FIG. 9B, a front plate that includes a first window area 611 and a second window area 612 (e.g., the front plate 510 of FIGS. 4 and 5, the front plate 610 of FIGS. 6 to 8) may be provided. A first conductive member (e.g., the first conductive member 721 of FIGS. 6, 7, 8, and 9A) may be formed on the front plate 610, or the first conductive member may not be formed on the front plate 610. After a second conductive material is deposited on an opposite surface 902 of the second window area 612, a second conductive member 722 may be formed on a specified portion of the opposite surface 902 of the second window area 612 by etching the second conductive material using a laser. The second conductive material may be formed of a same material as or a different material from the first conductive material. For example, the second conductive material may be formed in a single-layer or multi-layer structure that includes at least any one of Ti and Al. A third conductive member 723 may be formed on a specified portion of an opposite surface 902 of the second window area 612, on which the second conductive member 722 is formed, by printing a conductive paste. A third conductive member 723 may be formed on a specified portion of an opposite surface 902 of the second window area 612, on which the second conductive member 722 is formed, by printing a conductive paste. The protective member 810 may be formed to expose a partial area of the third conductive member 723 that contacts the conductive connection member 730. The display 620 (or a display panel) may be seated in the first window area 611. After the display 620 is seated, a first molding member 741 and a second molding member 742 may be formed through a molding process. After the first molding member 741 and the second molding member 742 are formed, a flexible printed circuit board (e.g., the flexible printed circuit board 750 of FIGS. 6, 7, and 8) may be fixed to the display 620. Then, the flexible printed circuit board and the third conductive member 723 exposed through the protective member 810 may be electrically connected to each other through a conductive connection member 730 formed by a jetting process.

According to an embodiment, in the wearable electronic device, the third conductive member 723 may be omitted. The second conductive member 722 may be formed in an area that contacts the conductive connection member 730 on an opposite surface 902 of the second window area 612. The conductive connection member 730 may directly contact the second conductive member 722 without the third conductive member 723.

According to an embodiment, after any one of the first conductive member 721 illustrated in FIG. 9A and the second conductive member 722 illustrated in FIG. 9B is formed first, a remaining one of the first conductive member 721 illustrated in FIG. 9A and the second conductive member 722 illustrated in FIG. 9B may be formed later. As an example, after the first conductive member 721 illustrated in FIG. 9A is formed first, the second conductive member 722 illustrated in FIG. 9B may be formed. As an example, after the second conductive member 722 illustrated in FIG. 9B is formed first, the first conductive member 721 illustrated in FIG. 9A may be formed.

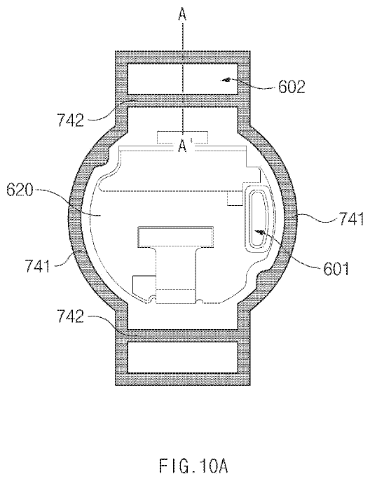

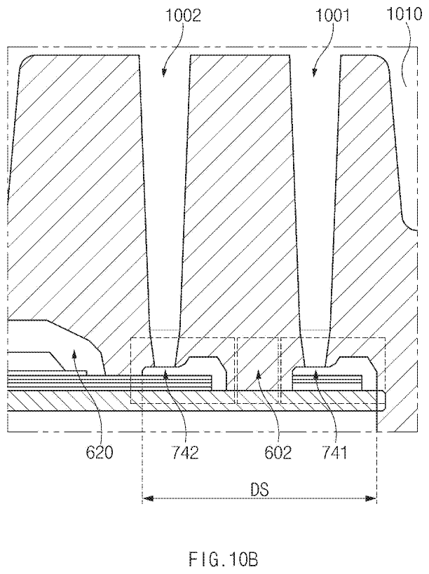

FIG. 10A is a view illustrating a molding member included in a wearable electronic device according to an embodiment of the disclosure.

FIG. 10B is a view illustrating an apparatus (e.g., a mold) for forming the molding member illustrated in FIG. 10A according to an embodiment of the disclosure.

Referring to FIGS. 10A and 10B, a wearable electronic device (e.g., the wearable electronic device 500 of FIGS. 4 and 5, the wearable electronic device 600 of FIGS. 6 and 7, the wearable electronic device 800 of FIG. 8) may include a first molding member 741 and a second molding member 742.

According to an embodiment, the first molding member 741 may be formed to surround at least a portion of a periphery of the first inner space 601 in which the display 620 is accommodated and/or at least a portion of a periphery of the second inner space 602 in which a sensor assembly (e.g., the sensor assembly 701 of FIGS. 6, 7, and 8) is accommodated. The second molding member 742 may be disposed between the first inner space 601 and the second inner space 602.

According to an embodiment, a portion of the first molding member 741 and/or at least a portion of the second molding member 742 may be formed at an outer periphery of the display 620. A portion of the first molding member 741 and/or at least a portion of the second molding member 742 may be formed to overlap a dead space DS (or a bezel, an inactive area, or a non-display area) of the display 620. When the display 620 is viewed from above, a portion of the first molding member 741 and/or at least a portion of the second molding member 742 may be formed to overlap a dead space DS of the display 620.

According to an embodiment, at least any one of the first molding member 741 and the second molding member 742 may be formed through injection-molding. As an example, the first molding member 741 and the second molding member 742 may be manufactured simultaneously in the same process using a same material or different materials. As an example, the first molding member 741 and the second molding member 742 may be manufactured in separate processes using a same material or different materials.

According to an embodiment, the first molding member 741 and the second molding member 742 may be formed using a mold 1010 illustrated in FIG. 10B. The mold 1010 may include a cap that has a first gate part 1001 and a second gate part 1002. As an example, the cap may be formed of a silicon material. The first gate part 1001 and the second gate part 1002 may be spaced apart from each other with the second inner space 602 interposed therebetween.

According to an embodiment, after the first gate part 1001 and the second gate part 1002 of the mold 1010 are aligned with an outer periphery of the display 620, a first molding member 741 and a second molding member 742 may be molded on the outer periphery of the display 620 by injecting an injection material into the first gate part 1001 and the second gate part 1002.

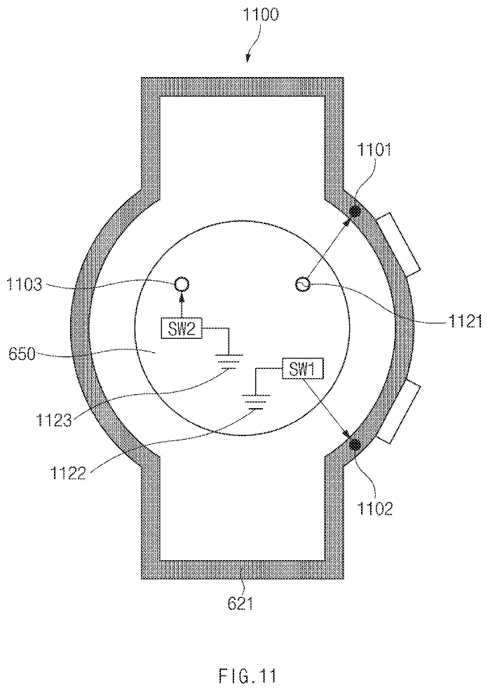

FIG. 11 is a view illustrating a portion of a wearable electronic device 1100 according to an embodiment of the disclosure.

Referring to FIG. 11, a wearable electronic device 1100 according to an embodiment may include a side member 621. The side member 621 may be formed in a loop shape that surrounds a circumference of the printed circuit board 650. The side member 621 may be physically and/or electrically connected to the printed circuit board 650 to form an antenna path. A portion of the side member 621 may function as a radiator of an antenna. For example, the side member 621, at least a portion of the side member 621 may be formed of a conductive material (e.g., metal). A conductive material portion of the side member 621 may form an electrical path through which an electrical signal may move to form a radiation pattern of a frequency band corresponding to the electrical path.

In an embodiment, a wireless communication device (e.g., the wireless communication module 192 of FIG. 1) may apply an electrical signal to the side member 621 through a power feeding part (feeder) 1121. For example, the wireless communication device may apply an electrical signal (e.g., an RF signal) to the side member 621 according to data received from a processor (e.g., the processor 120 of FIG. 1). The wireless communication device may transmit and receive an electrical signal corresponding to an electrical path formed in the side member 621.

According to an embodiment, at least one power feeding part 1121 and a plurality of ground parts 1122 and 1123 may be disposed on the printed circuit board 650.

The power feeding part 1121 may be electrically connected to the wireless communication device. In an embodiment, the power feeding part 1121 may be electrically connected to a power feeding point 1101 of the side member 621.

Each of the plurality of ground parts 1122 and 1123 may transmit an electrical signal applied to the side member 621 to ground. In an embodiment, each of the plurality of ground parts 1122 and 1123 may be connected to shorting points 1102 and 1103 of the side member 621, respectively, through switching elements SW1 and SW2. The first ground part 1122 may be connected to a first shorting point 1102 of the side member 621 through a first switching element SW1. An antenna loop may be formed between the first shorting point 1102 and the power feeding point 1101. The second ground part 1123 may be connected to a second shorting point 1103 of the side member 621 through a second switching element SW2.

The plurality of ground parts 1122 and 1123 may be selectively shorted with respect to the side member 621 through switching operations of the switching elements SW1 and SW2 to change an electrical path formed in the side member 621. In an embodiment, a shorting operation of the plurality of ground parts 1122 and 1123 may be determined according to a wireless communication mode of the wearable electronic device 1100.

According to an embodiment, at least any one of the power feeding point 1101 and the first shorting point 1102 may be formed at a portion of the side member 621 that surrounds the printed circuit board 650 and/or at a portion of the side member 621 that surrounds the sensor assembly 701. As an example, the power feeding point 1101 and the first shorting point 1102 may be formed at a portion of the side member 621 that surrounds the printed circuit board 650 or the sensor assembly 701. As an example, any one of the power feeding point 1101 and the first shorting point 1102 may be formed at a portion of the side member 621 that surrounds the printed circuit board 650, and a remaining one of the power feeding point 1101 and the first shorting point 1102 may be formed at a portion of the side member 621 that surrounds the sensor assembly 701.

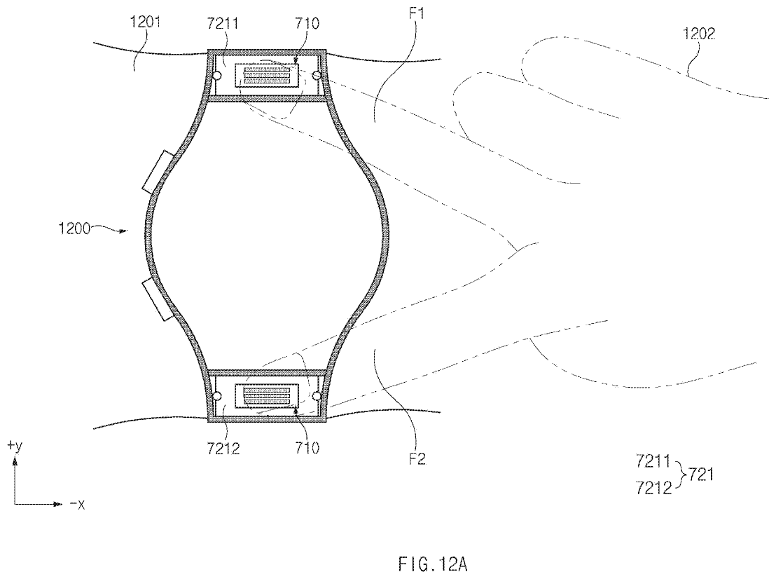

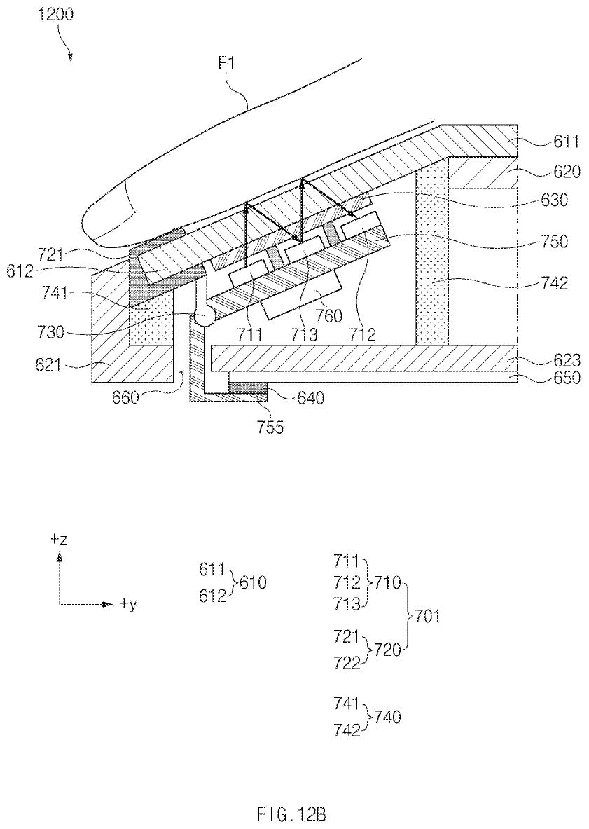

FIG. 12A is a view illustrating a state, in which a wearable electronic device 1200 is mounted on a body of a user according to an embodiment of the disclosure.

FIG. 12B is a conceptual view illustrating a method for acquiring biometric information through the wearable electronic device 1200 according to an embodiment of the disclosure.

In FIGS. 12A and 12B, the electronic device 1200 will be described based on a state in which the electronic device 1200 is worn on a left wrist 1201 of a user. In describing the embodiments illustrated in FIGS. 12A and 12B, for configurations that are identical to any one of the previous embodiments of FIGS. 1, 2A, 2B, to 8, 9A, 9B, 10A, 10B, and 11 or that may be easily understood from the previous embodiments, the same reference numerals are assigned or omitted, and detailed descriptions thereof are also omitted.

Referring to FIGS. 12A and 12B, a wearable electronic device 1200 according to an embodiment may include a second biometric sensor unit 720. The second biometric sensor unit 720 may include a first conductive member 721 that includes a first external electrode 7211 and a second external electrode 7212. The first external electrode 7211 and the second external electrode 7212 may be disposed to be spaced apart from each other with the display 620 interposed therebetween. The first external electrode 7211 may be disposed at any one of one side (or an upper side) or an opposite side (or a lower side) based on a direction facing the display 620. The second external electrode 7212 may be disposed at a remaining one of one side or an opposite side based on a direction that faces the display 620.

According to an embodiment, a front surface of the wearable electronic device 1200 may contact the right fingers F1 and F2 at least partially. The first external electrode 7211 and the second external electrode 7212, which partially form a front surface of the wearable electronic device (or the front plate 610), may contact a first finger F1 and a second finger F2 of a right hand 1202, respectively. When the right fingers F1 and F2 of a user contact the first external electrode 7211 and the second external electrode 7212, the electronic device 1200 may obtain biometric information of the user in a state in which the right hand 1202 of the user does not block at least a portion of the first window area 611 corresponding to the display 620. A rear surface of the wearable electronic device 1200 may contact the left wrist 1201 at least partially. The third external electrode (e.g., the first electrode area 213 of FIG. 2B) and the fourth external electrode (e.g., the second electrode area 214 of FIG. 2B), which partially form a rear surface of the wearable electronic device 1200, may contact different points of the left wrist. The wearable electronic device 1200 may identify biometric information of a user through an electrical loop formed through at least any one of the first external electrode 7211, the second external electrode 7212, the third external electrode, and the fourth external electrode. As an example, in a state in which a body of a user contacts the first external electrode 7211 and the second external electrode 7212, biometric information of the user may be identified through an electrical loop that is formed through the right fingers F1 and F2 of the user, the first external electrode 7211, the second external electrode 7212, and the left wrist 1201. As an example, in a state in which a body of a user contacts the first external electrode 7211, the second external electrode 7212, the third external electrode, and the fourth external electrode, biometric information of the user may be identified through an electrical loop that is formed through the right fingers F1 and F2 of the user, the first external electrode 7211, the second external electrode 7212, the third external electrode, the fourth external electrode, and the left wrist 1201.

According to an embodiment, the body of the user may contact at least any one of the first external electrode 7211, the second external electrode 7212, the third external electrode, and the fourth external electrode. At least any one of the first external electrode 7211, the second external electrode 7212, the third external electrode, and the fourth external electrode may be electrically connected to a processor (e.g., the processor 120 of FIG. 1). The processor may sense contact of the user with the first external electrode 7211, the second external electrode 7212, the third external electrode, and the fourth external electrode. The processor may obtain an electrical signal from a part of the body of the user through at least any one of the first external electrode 7211, the second external electrode 7212, the third external electrode, and the fourth external electrode, which contacts the body of the user. The processor may be configured to detect biometric information of the user based on the obtained electrical signal. As an example, when contact of the user with the first external electrode 7211, the second external electrode 7212, the third external electrode, and the fourth external electrode is sensed, the processor may obtain body impedance analysis (BIA) information of the user through the first external electrode 7211, the second external electrode 7212, the third external electrode, and the fourth external electrode. As an example, when contact of the user with any one of the first external electrode 7211 and the second external electrode 7212, and the third external electrode and the fourth external electrode is sensed, the processor may obtain an electrocardiogram (ECG) of the user through any one of the first external electrode 7211 and the second external electrode 7212, and the third external electrode and the fourth external electrode.

The wearable electronic device 1200 according to an embodiment may include a first biometric sensor unit 710. The first biometric sensor unit 710 may include a first light emitting unit 711, a second light emitting unit 712, and a light receiving unit 713. At least any one of the first light emitting unit 711 and the second light emitting unit 712 may generate light and may irradiate the light onto a finger F1 and F2 of a user that is in contact with the second window area 612 of the front plate 610. Light emitted by at least any one of the first light emitting unit 711 and the second light emitting unit 712 may be reflected by a finger of a user that is in contact with the second window area 612 of the front plate 610. Light reflected by a finger of a user may reach the light receiving unit 713 of the first biometric sensor unit 710. The light receiving unit 713 may obtain an optical signal that is reflected by fingers F1 and F2 of the user. A processor may be configured to detect biometric information of a user based on the obtained optical signal.

FIG. 13 is a conceptual view illustrating an example of a method for acquiring biometric information through a wearable electronic device 1300 according to an embodiment of the disclosure.

In FIG. 13, the wearable electronic device 1300 will be described based on a state in which the wearable electronic device 1300 is worn on a left wrist 1301 of a user. In describing the embodiments illustrated in FIG. 13, for configurations that are identical to any one of the previous embodiments of FIGS. 1, 2A, 2B, 8, 9A, 9B, 10A, 10B, to 12 or that may be easily understood from the previous embodiments, the same reference numerals are assigned or omitted, and detailed descriptions thereof are also omitted.

Referring to FIG. 13, the electronic device may identify biometric information (e.g., body impedance analysis (BIA)) of a user by using two key buttons 1310 and 1320 (e.g., key buttons 203 and 204 of FIG. 2A) that are disposed on a portion of a side surface of the electronic device.

According to an embodiment, the electronic device 1300 may identify an electrical loop that is formed through at least one of two key buttons 1310 and 1320 that are disposed on a portion of a side surface of the electronic device, a first electrode area (e.g., a first electrode area 213 of FIG. 2B) that is disposed on a rear plate, and a second electrode area (e.g., a second electrode area 214 of FIG. 2B) that is disposed on the rear plate, and may identify BIA information of a user based on a BIA signal that is obtained based on the electrical loop.

According to an embodiment, the body (e.g., fingers F1 and F2 of a right hand) of the user may contact at least one of the key buttons 1310 and 1320, the first electrode area, and the second electrode area. At least one of the key buttons 1310 and 1320, the first electrode area, and the second electrode area may be electrically connected to a processor (e.g., a processor 120 of FIG. 1). A processor may sense contact of the user with at least one of the key buttons 1310 and 1320, the first electrode area, and the second electrode area. A processor may obtain an electrical signal from a part of the body of the user through at least one of the key buttons 1310 and 1320, the first electrode area, and the second electrode area, with which the body of the user is in contact. The processor may be configured to detect biometric information of the user based on the obtained electrical signal. As an example, when contact of the user with the key buttons 1310 and 1320 is sensed, a processor may obtain biometric information of the user through the key buttons 1310 and 1320.

Meanwhile, the above-described wearable electronic device may selectively turn on (or activate) a part of the plurality of electrodes according to a wearing type of the user and/or a contact type with the body of the user, or may display guide information for measuring biometric information, thereby providing intuitive and convenient usability.

Meanwhile, the above-described wearable electronic device is not limited to the embodiments described in the drawings, and the wearable electronic devices described in the drawing may be applied in a complex manner. Furthermore, a position, a number, and a shape of a second inner space 602, in which the sensor assembly 701 described in each drawing is disposed, may be variously changed without being limited to the above-described example. Furthermore, a number and a shape of at least one of the light emitting units 711 and 712, the light receiving unit 713, the first conductive member 721, the second conductive member 722, and the conductive connection member 730 described in each drawing may be variously changed without being limited to the above-described example. As an example, structures of the first light emitting unit 711, the second light emitting unit 712, and the light receiving unit 713 are not limited to structures of FIG. 12A and/or FIG. 13 and may be variously changed.

As described above, a wearable electronic device according to at least one of various embodiments may include a housing 501 that includes a front plate 510 and 610, a portion of which is formed to be inclined, a rear plate 522 and 622 that faces a direction opposite to the front plate 510 and 610, and a side member 521 and 621 that surrounds an inner space 503 between the front plate 510 and 610 and the rear plate 522 and 622, a molding member 740 that is disposed between the front plate 510 and 610 and the rear plate 522 and 622 and divides the inner space 503 into a first inner space 601 and a second inner space 602, a display 620 that is disposed in the first inner space 601, and a sensor assembly 701 that does not overlap the display 620 and is disposed in the second inner space 602.

According to an embodiment, the front plate 510 and 610 may be formed to cover the display 620 and the sensor assembly 701.

According to an embodiment, the front plate 510 and 610 may include a first window area 511 and 611 that covers the display 620, and a second window area 512 and 612 that is integrally formed with the first window area 511 and 611, at least a portion of which has an inclined surface, and that covers the sensor assembly 701.

According to an embodiment, the second window area 512 and 612 may be formed to be inclined from the side member 521 and 621 toward the first window area 511 and 611.

According to an embodiment, the second window area 512 and 612 may form an obtuse angle with the first window area 511 and 611.

According to an embodiment, at least a portion of the front plate 510 and 610 may face a first direction (+z), and at least a portion of the rear plate 522 and 622 may face a second direction (−z), which is opposite to the first direction (+z).

According to an embodiment, the second window area 512 and 612 may extend from the first window area 511 and 611 in a third direction (+y, −y), which is perpendicular to the first direction (+z) and the second direction (−z).

According to an embodiment, the electronic device may further include a fastening member 595 and 597 that is connected to the housing 501.

According to an embodiment, the second window area 512 and 612 may extend from the first window area 511 and 611 toward the fastening member 595 and 597.

According to an embodiment, the electronic device may further include a flexible printed circuit board 750 in which a portion of the sensor assembly 701 is disposed, and a printed circuit board 650 that is electrically connected to the flexible printed circuit board 750, the display 620, and the sensor assembly 701.

According to an embodiment, at least a portion of the flexible printed circuit board 750 may be disposed in parallel to the second window area 512 and 612.

According to an embodiment, at least a portion of the flexible printed circuit board 750 may be disposed to have the same inclination angle as the second window area 512 and 612.

According to an embodiment, the molding member 740 may include a first molding member 741 that is formed to at least partially contact an inside of the side member 621 and that is formed along the side member 621, and a second molding member 742 that is disposed in parallel to the side member between the display 620 and the sensor assembly 701.

According to an embodiment, the electronic device may further include a bracket 623 that is disposed between the printed circuit board 650 and the front plate 510 and 610.

According to an embodiment, the second molding member 742 may be disposed between the front plate 510 and 610 and the bracket 623.

According to an embodiment, the sensor assembly 701 may be disposed at opposite sides of the display 620.

According to an embodiment, the sensor assembly 701 may include a first biometric sensor unit 710 that is disposed in the second inner space 602, and a second biometric sensor unit 720 that is disposed to surround the first biometric sensor unit 710.

According to an embodiment, the first biometric sensor unit 710 may include at least one light receiving unit 713 and at least one light emitting unit 711 and 712.

According to an embodiment, the electronic device may further include an optical film 630 that is disposed between each of the at least one light receiving unit 713 and the at least one light emitting unit 711 and 712, and the second window area 512 and 612.

According to an embodiment, the second biometric sensor unit 720 may include a first conductive member 721 that is disposed outside the second inner space 602 and is disposed on one surface 901 of the front plate 510 and 610, and a second conductive member 722 that is electrically connected to the first conductive member 721 and is disposed on an opposite surface 902 of the front plate 510 and 610 in the second inner space 602.

According to an embodiment, the electronic device may further include a conductive connection member 730 that is disposed between the second conductive member 722 and the flexible printed circuit board 750 and that electrically connects the second conductive member 722 and the flexible printed circuit board 750.

A wearable electronic device according to at least one of various embodiments may include a housing 501 that includes a front plate 510 and 610, a portion of which is formed to be inclined, a display 620 that is covered by the front plate 510 and 610 and is disposed in the housing 501, a sensor assembly 701 that is covered by the front plate 510 and 610 and is disposed adjacent to the display 620, and a molding member that is disposed between the display 620 and the sensor assembly 701.

According to an embodiment, the sensor assembly 701 may include a first biometric sensor unit 710 that does not overlap the display 620 and is disposed in the housing 501, and a second biometric sensor unit 720 that includes a first conductive member 721 that does not overlap the display 620 and is partially formed on one surface 901 of the front plate 510 and 610.

According to an embodiment, the front plate 510 and 610 may include a first window area 511 and 611 that covers the display 620, and a second window area 512 and 612 that is integrally formed with the first window area 511 and 611, at least a portion of which is formed to be inclined, and that covers the sensor assembly 701.

According to an embodiment, the wearable electronic device may further include a flexible printed circuit board 750 in which a portion of the sensor assembly 701 is disposed.

According to an embodiment, at least a portion of the flexible printed circuit board 750 may be disposed in parallel to the second window area 512 and 612.

According to an embodiment, the first biometric sensor unit 710 may include at least one light receiving unit 713 and at least one light emitting unit 711 and 712.

According to an embodiment, the second biometric sensor unit 720 may further include a second conductive member 722 that is electrically connected to the first conductive member 721 and is disposed on an opposite surface 902 of the front plate 510 and 610 in the housing 501.

According to an embodiment, the first conductive member 721 may be disposed outside the housing 501 and may be disposed on one surface 901 of the front plate 510 and 610.

According to an embodiment, the molding member 740 may include a first molding member 741 that is formed along a portion of a circumference of the display 620 and a portion of a circumference of the sensor assembly 701, and a second molding member 742 that is connected to the first molding member 741 and is disposed between the display 620 and the sensor assembly 701.

An embodiment of disclosure and terms used herein are not intended to limit the technologies described in the disclosure to specific embodiments, and it should be understood that the embodiments and the terms include modification, equivalent, and/or alternative on the corresponding embodiment described herein. With regard to the description of drawings, similar components may be marked by similar reference marks/numerals. In the disclosure, the expressions “A or B”, “at least one of A and/or B”, “A, B, or C”, or “at least one of A, B, and/or C”, and the like used herein may include any and all combinations of one or more of the associated listed items. Expressions such as “first,” or “second,” and the like, may express their components regardless of their priority or importance and may be used to distinguish one component from another component but is not limited to these components. When a certain component (e.g., a first component) is referred to as being “connected” or “coupled” (functionally or communicatively) to another component (e.g., a second component), it should be understood that the certain component may be directly connected to the other component or may be connected to the other component through another component (e.g., a third component).

According to the situation, the expression “adapted to or configured to” used herein may be interchangeably used as, for example, the expression “suitable for”, “having the capacity to”, “changed to”, “made to”, “capable of” or “designed to”. The expression “a device configured to” may mean that the device is “capable of” operating together with another device or other parts. For example, a “processor configured to (or set to) perform A, B, and C” may mean a dedicated processor (e.g., an embedded processor) for performing corresponding operations or a generic-purpose processor (e.g., a central processing unit (CPU) or an AP) which performs corresponding operations by executing one or more software programs which are stored in a memory device (e.g., the memory).

The term “module” used herein may include a unit, which is implemented with hardware, software, or firmware, and may be interchangeably used with the terms “logic”, “logical block”, “part”, “circuit”, or the like. The “module” may be an integrated component, a minimum unit for performing one or more functions, or a part thereof. The “module” may be implemented mechanically or electronically. For example, the module may include a well-known or to-be-developed application-specific integrated circuit (ASIC) chip, field-programmable gate arrays (FPGAs), or programmable logic device that perform any operations.

According to various embodiment, at least part of a device (e.g., modules or functions thereof) or a method (e.g., operations) may be, for example, implemented by instructions stored in a computer-readable storage media (e.g., a memory) in the form of a program module. The instruction, when executed by a processor (e.g., a processor), may cause the processor to perform a function corresponding to the instruction. The computer-readable recording medium may include a hard disk, a floppy disk, a magnetic media (e.g., a magnetic tape), an optical medium (e.g., a compact disc read only memory (CD-ROM) and a digital versatile disc (DVD), a magneto-optical media (e.g., a floptical disk)), an embedded memory, or the like. The one or more instructions may contain a code made by a compiler or a code executable by an interpreter.

Each element (e.g., a module or a program module) according to various embodiments may be composed of single entity or a plurality of entities, a part of the above-described sub-elements may be omitted or may further include other elements. Alternatively or additionally, some components (e.g., a module or a program module) may be combined with each other so as to form one entity, so that the functions of the components may be performed in the same manner as before the combination. According to various embodiments, operations executed by modules, program modules, or other components may be executed by a successive method, a parallel method, a repeated method, or a heuristic method. Alternatively, at least some of the operations may be executed in another order or may be omitted, or any other operation may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a housing including:
a front plate being partially inclined,
a rear plate facing in a direction opposite to the front plate, and
a side member surrounding an inner space between the front plate and the rear plate;
a molding member disposed between the front plate and the rear plate, the molding member dividing the inner space into a first inner space and a second inner space;
a display disposed in the first inner space; and
a sensor assembly non-overlapping with the display, the sensor assembly being disposed in the second inner space,
wherein the front plate covers the display and the sensor assembly.

2. The electronic device of claim 1, wherein the front plate includes:
a first window area covering the display, and
a second window area disposed integrally with the first window area, at least a portion of which has an inclined surface, the second window area covering the sensor assembly.

3. The electronic device of claim 2,
wherein the second window area is inclined from the side member toward the first window area, and
wherein the second window area forms an obtuse angle with the first window area.

4. The electronic device of claim 2,
wherein at least a portion of the front plate faces a first direction,
wherein the direction opposite to the front plate is a second direction, the second direction being opposite to the first direction, and
wherein the second window area extends from the first window area in a third direction perpendicular to the first direction and the second direction.

5. The electronic device of claim 2, further comprising:
a fastening member connected to the housing,
wherein the second window area extends from the first window area toward the fastening member.

6. The electronic device of claim 2, further comprising:
a flexible printed circuit board on which a portion of the sensor assembly is disposed; and
a printed circuit board electrically connected to the flexible printed circuit board, the display, and the sensor assembly.

7. The electronic device of claim 6, wherein at least a portion of the flexible printed circuit board is disposed parallel to the second window area.

8. The electronic device of claim 6, wherein at least a portion of the flexible printed circuit board is disposed to have a same inclination angle as the second window area.

9. The electronic device of claim 6, wherein the molding member includes:
a first molding member disposed along the side member, the first molding member being at least partially in contact with an inside of the side member, and
a second molding member disposed in parallel with the side member between the display and the sensor assembly.

10. The electronic device of claim 9, further comprising:
a bracket disposed between the printed circuit board and the front plate,
wherein the second molding member is disposed between the front plate and the bracket.

11. The electronic device of claim 6,
wherein the sensor assembly is disposed on both sides of the display,
wherein the sensor assembly includes:
a first biometric sensor unit disposed in the second inner space, and
a second biometric sensor unit disposed to surround the first biometric sensor unit within the second inner space.

12. The electronic device of claim 11, wherein the first biometric sensor unit includes at least one light receiving unit and at least one light emitting unit.

13. The electronic device of claim 12, further comprising:
an optical film disposed between the second window area and each of (i) the at least one light receiving unit and (ii) the at least one light emitting unit.

14. The electronic device of claim 11, wherein the second biometric sensor unit includes:
a first conductive member disposed outside the second inner space, the first conductive member being disposed on a first surface of the front plate; and
a second conductive member electrically connected to the first conductive member, the second conductive member being disposed on a second surface of the front plate within the second inner space.

15. The electronic device of claim 14, further comprising:
a conductive connection member disposed between the second conductive member and the flexible printed circuit board to electrically connect the second conductive member and the flexible printed circuit board.

16. A wearable electronic device comprising:
a housing including a front plate, a portion of which is inclined;
a display covered by the front plate, the display being disposed within the housing;
a sensor assembly covered by the front plate, the sensor assembly being disposed adjacent to the display; and
a molding member disposed between the display and the sensor assembly,
wherein the sensor assembly includes:
a first biometric sensor unit disposed within the housing, the first biometric sensor unit non-overlapping with the display, and
a second biometric sensor unit non-overlapping with the display, the second biometric sensor unit including a first conductive member partially disposed on a first surface of the front plate.

17. The wearable electronic device of claim 16, wherein the front plate includes:
a first window area covering the display; and
a second window area disposed integrally with the first window area, at least a portion of which has an inclined surface, the second window area covering the sensor assembly.

18. The wearable electronic device of claim 17, further comprising:
a flexible printed circuit board on which a portion of the sensor assembly is disposed,
wherein at least a portion of the flexible printed circuit board is disposed parallel to the second window area.

19. The wearable electronic device of claim 16,
wherein the first biometric sensor unit includes at least one light receiving unit and at least one light emitting unit,
wherein the second biometric sensor unit further includes a second conductive member electrically connected to the first conductive member, the second conductive member being disposed on a second surface of the front plate within the housing, and
wherein the first conductive member is disposed outside the housing, the first conductive member being disposed on the first surface of the front plate.

20. The wearable electronic device of claim 19, wherein the molding member includes:
a first molding member disposed along a portion of a periphery of the display and a portion of a periphery of the sensor assembly, and
a second molding member connected to the first molding member, the second molding member being disposed between the display and the sensor assembly.
